(19) United States
(12) Patent Application Publication
(10) Pub. No.: US 2022/0277828 A1
(43) Pub. Date: Sep. 1, 2022

(54) METHODS AND SYSTEMS FOR REVERSING A GERIATRIC PROCESS

(71) Applicant: KPN INNOVATIONS, LLC., Lakewood, CO (US)

(72) Inventor: Kenneth Neumann, LAKEWOOD, CO (US)

(73) Assignee: KPN INNOVATIONS, LLC., Lakewood, CO (US)

(21) Appl. No.: 17/187,975

(22) Filed: Mar. 1, 2021

(51) Int. Cl. G16H 20/60; G06N 20/00

(52) U.S. Cl. CPC G16H 20/60; G06N 20/00

(57) ABSTRACT

A system for reversing a geriatric process, the system including a computing device configured to receive, a geriatric process relating to a user; retrieve a user effective age; assign the geriatric process a geriatric group label as a function of the user effective age; identify a measurement relating to the geriatric group label; and locate a nutrient intended to address the measurement, wherein locating the nutrient includes training a machine learning process as a function of a training set, wherein the training set relates a plurality of measurements to a plurality of nutrients; and locating the nutrient as a function of the measurement and the machine learning process.

### FIELD OF THE INVENTION

The present invention generally relates to the field of artificial intelligence. In particular, the present invention is directed to methods and systems for reversing a geriatric process.

### BACKGROUND

Geriatric processes can be difficult to ascertain. Frequently, they may present with other complex challenges. Identifying custom nutrients can be difficult particularly when coupled with other maladies.

### SUMMARY OF THE DISCLOSURE

In an aspect, a system for reversing a geriatric process, the system comprising a computing device the computing device designed and configured to receive, a geriatric process relating to a user; retrieve a user effective age; assign the geriatric process a geriatric group label as a function of the user effective age; identify a measurement relating to the geriatric group label; and locate a nutrient intended to address the measurement, wherein locating the nutrient further comprises training a machine learning process as a function of a training set, wherein the training set relates a plurality of measurements to a plurality of nutrients; and locating the nutrient as a function of the measurement and the machine learning process.

In an aspect, a method of reversing a geriatric process, the method comprising receiving by a computing device, a geriatric process relating to a user; retrieving by the computing device, a user effective age; assigning the geriatric process, by the computing device, a geriatric group label as a function of the user effective age; identifying by the computing device, a measurement relating to the geriatric group label; and locating by the computing device, a nutrient intended to address the measurement, wherein locating the nutrient further comprises training a machine learning process as a function of a training set, wherein the training set relates a plurality of measurements to a plurality of nutrients; and locating the nutrient as a function of the measurement and the machine learning process.

These and other aspects and features of non-limiting embodiments of the present invention will become apparent to those skilled in the art upon review of the following description of specific non-limiting embodiments of the invention in conjunction with the accompanying drawings.

### BRIEF DESCRIPTION OF THE DRAWINGS

For the purpose of illustrating the invention, the drawings show aspects of one or more embodiments of the invention. However, it should be understood that the present invention is not limited to the precise arrangements and instrumentalities shown in the drawings, wherein:

FIG. 1 is a block diagram illustrating an exemplary embodiment of a system for reversing a geriatric process;

FIG. 2 is a block diagram illustrating an exemplary embodiment of a user database;

FIG. 3 is a block diagram illustrating an exemplary embedment of a nutrient database;

FIG. 4 is a diagrammatic representation of a geriatric classifier;

FIG. 5 is a block diagram illustrating an exemplary embodiment of a machine learning module;

FIG. 6 is a process flow diagram illustrating an exemplary embodiment of a method of reversing a geriatric process; and

FIG. 7 is a block diagram of a computing system that can be used to implement any one or more of the methodologies disclosed herein and any one or more portions thereof.

The drawings are not necessarily to scale and may be illustrated by phantom lines, diagrammatic representations and fragmentary views. In certain instances, details that are not necessary for an understanding of the embodiments or that render other details difficult to perceive may have been omitted.

### DETAILED DESCRIPTION

At a high level, aspects of the present disclosure are directed to systems and methods for reversing a geriatric process. In an embodiment, a computing device receives a geriatric process and retrieves information pertaining to a user's effective age. A computing device assigns a geriatric group label and locates a nutrient intended to address a measurement. A nutrient may include a recommended meal intended to aid in mitigating a geriatric process.

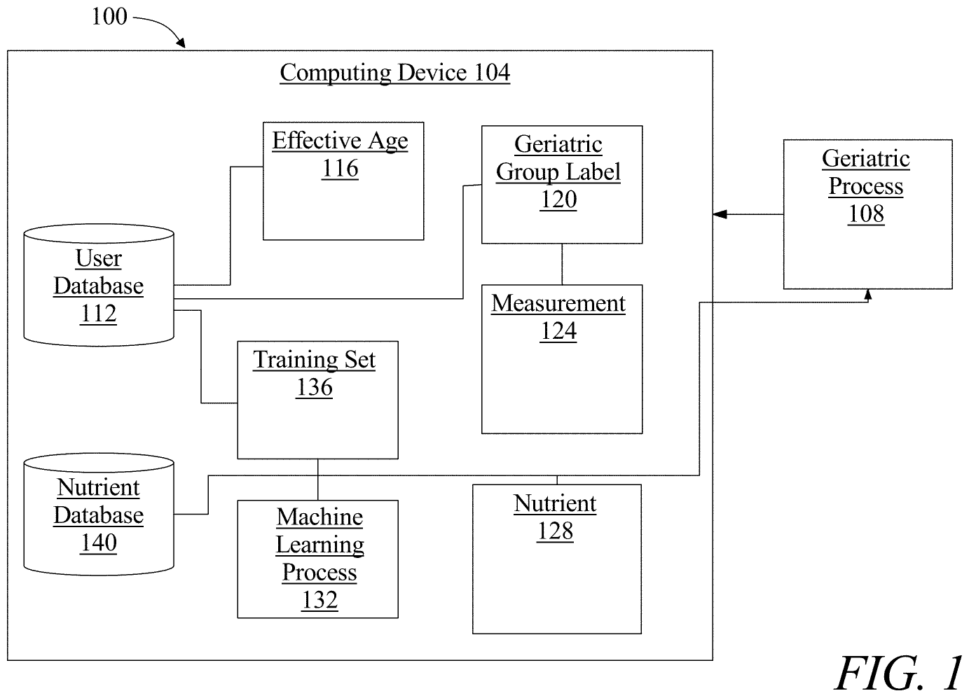

Referring now to the drawings, FIG. 1 illustrates an exemplary embodiment of a system 100 for reversing a geriatric process. System 100 includes a computing device 104. Computing device 104 may include any computing device as described in this disclosure, including without limitation a microcontroller, microprocessor, digital signal processor (DSP) and/or system on a chip (SoC) as described in this disclosure. Computing device 104 may include, be included in, and/or communicate with a mobile device such as a mobile telephone or smartphone. Computing device 104 may include a single computing device operating independently, or may include two or more computing device operating in concert, in parallel, sequentially or the like; two or more computing devices may be included together in a single computing device or in two or more computing devices. Computing device 104 may interface or communicate with one or more additional devices as described below in further detail via a network interface device. Network interface device may be utilized for connecting computing device 104 to one or more of a variety of networks, and one or more devices. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus, or other relatively small geographic space), a telephone network, a data network associated with a telephone/voice provider (e.g., a mobile communications provider data and/or voice network), a direct connection between two computing devices, and any possibilities thereof. A network may employ a wired and/or a wireless mode of communication. In general, any network topology may be used. Information (e.g., data, software etc.) may be communicated to and/or from a computer and/or a computing device. Computing device 104 may include but is not limited to, for example, a computing device or cluster of computing devices in a first location and a second computing device or cluster of computing devices in a second location. Computing device 104 may include one or more computing devices dedicated to data storage, security, distribution of traffic for load balancing, and the like. Computing device 104 may distribute one or more computing tasks as described below across a plurality of computing devices of computing device, which may operate in parallel, in series, redundantly, or in any other manner used for distribution of tasks or memory between computing devices. Computing device 104 may be implemented using a “shared nothing” architecture in which data is cached at the worker, in an embodiment, this may enable scalability of system 100 and/or computing device.

With continued reference to FIG. 1. computing device 104 may be designed and/or configured to perform any method, method step, or sequence of method steps in any embodiment described in this disclosure, in any order and with any degree of repetition. For instance, computing device 104 may be configured to perform a single step or sequence repeatedly until a desired or commanded outcome is achieved; repetition of a step or a sequence of steps may be performed iteratively and/or recursively using outputs of previous repetitions as inputs to subsequent repetitions, aggregating inputs and/or outputs of repetitions to produce an aggregate result, reduction or decrement of one or more variables such as global variables, and/or division of a larger processing task into a set of iteratively addressed smaller processing tasks. Computing device 104 may perform any step or sequence of steps as described in this disclosure in parallel, such as simultaneously and/or substantially simultaneously performing a step two or more times using two or more parallel threads, processor cores, or the like; division of tasks between parallel threads and/or processes may be performed according to any protocol suitable for division of tasks between iterations. Persons skilled in the art, upon reviewing the entirety of this disclosure, will be aware of various ways in which steps, sequences of steps, processing tasks, and/or data may be subdivided, shared, or otherwise dealt with using iteration, recursion, and/or parallel processing.

With continued reference to FIG. 1, computing device 104 is configured to receive a geriatric process 108, relating to a user. A “geriatric process,” as used in this disclosure, is an age-related disease, sickness, illness, infection, ailment, malady, disorder, complaint, affliction, condition, problem and the like. For instance, and without limitation, a geriatric process 108 may include a diagnosed medical condition such as urinary incontinence. In yet another non-limiting example, a geriatric process 108 may include a description of one or more symptoms that a user may be experiencing such as knee pain when standing. A geriatric process may include a description of a condition that may be associated with disability and difficulty in performing normal activities of daily living such as bathing, dressing, eating, and going to the bathroom. A geriatric process may include a description of a condition associated with aging such as a loss of mental sharpness, falls incontinence, dizziness, vision, and/or hearing problems.

With continued reference to FIG. 1, information pertaining to a geriatric process 108 may be stored within user database 112. User database 112 may be implemented, without limitation, as a relational database, a key-value retrieval datastore such as a NOSQL database, or any other format or structure for use as a datastore that a person skilled in the art would recognize as suitable upon review of the entirety of this disclosure. User database 112 may contain one or more entries including one or more geriatric processes 108 relating to a user.

With continued reference to FIG. 1, computing device 104 is configured to retrieve a user effective age 116. A “user effective age,” as used in this disclosure, is an age of a user adjusted to reflect a life expectancy that differs from an actuarially projected life expectancy. For instance, a user effective age of a person predicted to life fewer years than actuarially projected may be higher than a user effective age of a person predicted to match and/or exceed an actuarially projected life expectancy. User effective age 116 may be used as a representation of a user's likely overall state of health, inasmuch as a user's likelihood to exceed or fall short of actuarially projected life expectancy may be closely linked to a user's state of health. User effective age 116 may include any user effective age 116 as described in U.S. Nonprovisional application Ser. No. 16/558,502, filed on Sep. 3, 2019, and entitled “SYSTEMS AND METHODS FOR SELECTING AN INTERVENTION BASED ON EFFECTIVE AGE” the entirety of which is incorporated herein by reference. In an embodiment, a user effective age 116 may be calculated by multiplying a genetic factor by a lifestyle factor. A “genetic factor,” as used in this disclosure, is a result indicating any hereditary and inherited characteristics. For instance, and without limitation, a genetic factor may indicate that a user has an inherited DNA mutation such as a BRCA2 mutation. In yet another non-limiting example, a genetic factor may indicate that a user does not have a methylenetetrahydrofolate reductase (MTHFR) mutation. Information pertaining to a genetic factor may be stored within user database 112. A “lifestyle factor,” as used in this disclosure, is a result indicating a user's values, knowledge, and/or norms as shaped by cultural and/or socioeconomic factors and/or practices. A lifestyle factor may relate to behaviors, spirituality, physical fitness, social relationships, emotional support, mental well being and the like. For instance, and without limitation, a lifestyle factor may describe quantities of physical fitness that a user participates in on a weekly basis. In yet another non-limiting example, a lifestyle factor may indicate if a user participates in a meditation practice. Information pertaining to a lifestyle factor may be stored within user database 112.

With continued reference to FIG. 1, computing device 104 may subtract a user effective age 116 from a user chronological age and output a deficit. A “user chronological age,” as used in this disclosure, is an age of the user is an age of the user as measured in years, or other units of time, from the date of the user's birth to the date of the measurement, where a “date” may include any calendar date, Julian date, or the like. A user chronological age and a user effective age 116 may be used to calculate a deficit. A “deficit,” as used in this disclosure, is any difference between a user a user effective age 116 subtracted from a user chronological age. Computing device 104 may assign a geriatric group label as a function of a deficit, as described below in more detail.

With continued reference to FIG. 1, computing device 104 assigns a geriatric process 108 a geriatric group label 120 as a function of a user effective age 116. A “geriatric group label,” as used in this disclosure, is a label identifying a root cause of a geriatric process 108. A geriatric group label 120 may identify a body system involved and/or affected by s geriatric process 108. A body system may include a group of organs and/or tissues that may work together to perform important jobs for the body. Some organs may be part of more than one body system. Body systems may include but are not limited to the digestive system, muscular system, integumentary system, lymphatic system, endocrine system, nervous system, skeletal system, reproductive system, respiratory system, urinary system, circulatory system, and the like. A “root cause,” as used in this disclosure, is an initiating cause and/or a causal cause of a geriatric process 108. For instance, and without limitation, a geriatric group label 120 may identify that a geriatric process 108 such as dementia may have one or more root causes such as oxidative stress and/or small intestinal bacterial overgrowth (SIBO). In yet another non-limiting example, a geriatric group label 120 may identify that a geriatric process 108 such as recurring falls and injuries associated with falls may be due to one or more root causes such as dehydration and/or hypotension. In an embodiment, a geriatric group label 120 may identify that a user with a geriatric process 108 such as Information pertaining to geriatric group labels may be collected from expert medical and scientific journals, clinical experts, and the like and stored in a database as described below in more detail.

With continued reference to FIG. 1, computing device 104 may be configured to assign a geriatric group label 120 using a deficit as described above in more detail. For instance, and without limitation, a user with a user effective age 116 which indicates the user is forty-two years old, may have a chronological age of sixty-seven years of age, which may produce a deficit of twenty-five years of age. In such an instance, computing device 104 may assign a geriatric group label 120 using the twenty-five years of age deficit in conjunction with a geriatric process relating to a user. For instance, and without limitation, a first user with a geriatric process 108 such as hypothyroidism with a deficit of sixteen years may be assigned to a geriatric group label 120 such as selenium deficiency, while a second user with a geriatric process 108 such as hypothyroidism with a deficient of negative ten years may be assigned to a geriatric group label 120 such as autoimmune thyroiditis.

With continued reference to FIG. 1, computing device 104 may locate a malady state associated with a user. A “malady state,” as used in this disclosure, is a disease and/or ailment that the user may be afflicted and/or diagnosed with. For instance, and without limitation, a malady state may indicate that a user suffers from a chronic medical condition such as cardiovascular disease. In yet another non-limiting example, a malady state may indicate that a user had been previously diagnosed with chronic bronchitis. Information pertaining to a malady state may be stored within user database 112. Computing device 104 may compare a malady state to a geriatric process 108. For instance, and without limitation, computing device 104 may compare a malady state such as hypertension to a geriatric process 108 such as angina to ascertain a common body system and/or root cause. In such an instance, computing device 104 may utilize the comparison to assign a geriatric group label 120 such as cardiovascular disease.

With continued reference to FIG. 1, computing device 104 identifies a measurement 124 relating to a geriatric group label 120. A “measurement,” as used in this disclosure, is an imbalance associated with a user's constitution. A measurement 124 may include one or more physical measurements. A “physical measurement,” as used in this disclosure, is a measurement obtained from a physiological extraction. A physiological extraction may be obtained from a biofluid and/or specimen such as but not limited to a blood sample, a saliva sample, a urine sample, a stool sample, a hair sample, a tissue sample, a skin sample, a nail sample, a fixed and/or stained slide containing a human component such as a cervical sample and the like. A physiological extraction may be obtained from one or more blood components, including but not limited to peripheral blood, umbilical cord blood, serum blood, plasma blood, buffy coat blood and the like. A physiological extraction may be obtained from human primary cells derived from human bio samples, A physiological extraction may be obtained from deoxyribonucleic acid (DNA), ribonucleic acid (RNA), and/or any component of genetic material including but not limited to a gene, and/or a group of genes. A physiological extraction may be obtained from one or more cells of the human body, such as for example a stem cell. A physical measurement may include the results of one or more surveys and/or questionnaires completed by a user detailing a user's response to one or more questions and/or prompts for information. Information pertaining to a measurement 124 may be stored and contained within user database 112.

With continued reference to FIG. 1, measurement 124 may identify a deficiency. A “deficiency,” as used in this disclosure, is an imbalance caused by a lack or shortage. For instance, and without limitation, a measurement 124 may identify that a user has a Vitamin D deficiency. In yet another non-limiting example, a measurement 124 may identify that a user has a deficiency of essential fatty acids. Information pertaining to a deficiency may be stored within user database 112. In an embodiment, measurement 124 may identify an excess. An “excess,” as used in this disclosure, is an imbalance caused by exceeding a prescribed and/or desirable amount. For instance, and without limitation, a measurement 124 may identify that a user has an excess of iron in the blood. In yet another non-limiting example, a measurement 124 may identify that a user has an excess of low-density lipoprotein (LDL) in the user's blood.

With continued reference to FIG. 1, computing device 104 may identify a measurement 124 using a geriatric classifier. Geriatric classifier may be configured to input a geriatric process 108 relating to a user and output a measurement 124. A “classifier,” as used in this disclosure is a machine learning model, such as a mathematical model, neural net, or program generated by a machine learning algorithm known as a “classification algorithm,” as described in further detail below, that sorts inputs into categories or bins of data, outputting the categories or bins of data and/or labels associated therewith. A classifier may be configured to output at least a datum that labels or otherwise identifies a set of data that are clustered together, found to be close under a distance metric as described below, or the like. Computing device 104 and/or another device may generate a classifier using a classification algorithm, defined as a process whereby a computing device 104 derives a classifier from training data. Geriatric classifier may be trained using training data that may contain a plurality of data entries relating geriatric processes to a plurality of measurements. Computing device 104 may train classifier utilizing training data. Training data may be obtained from expert input, previous iterations of generating classifier, and/or from publicly available sources. Classification may be performed using, without limitation, linear classifiers such as without limitation logistic regression and/or naive Bayes classifiers, nearest neighbor classifiers such as k-nearest neighbors' classifiers, support vector machines, least squares support vector machines, fisher's linear discriminant, quadratic classifiers, decision trees, boosted trees, random forest classifiers, learning vector quantization, and/or neural network-based classifiers.

Still referring to FIG. 1, computing device 104 may be configured to generate a classifier using a Naïve Bayes classification algorithm. Naïve Bayes classification algorithm generates classifiers by assigning class labels to problem instances, represented as vectors of element values. Class labels are drawn from a finite set. Naïve Bayes classification algorithm may include generating a family of algorithms that assume that the value of a particular element is independent of the value of any other element, given a class variable. Naïve Bayes classification algorithm may be based on Bayes Theorem expressed as P(A/B)=P(B/A) P(A)÷P(B), where P(AB) is the probability of hypothesis A given data B also known as posterior probability; P(B/A) is the probability of data B given that the hypothesis A was true; P(A) is the probability of hypothesis A being true regardless of data also known as prior probability of A; and P(B) is the probability of the data regardless of the hypothesis. A naïve Bayes algorithm may be generated by first transforming training data into a frequency table. Computing device 104 may then calculate a likelihood table by calculating probabilities of different data entries and classification labels. Computing device 104 may utilize a naïve Bayes equation to calculate a posterior probability for each class. A class containing the highest posterior probability is the outcome of prediction. Naïve Bayes classification algorithm may include a gaussian model that follows a normal distribution. Naïve Bayes classification algorithm may include a multinomial model that is used for discrete counts. Naïve Bayes classification algorithm may include a Bernoulli model that may be utilized when vectors are binary.

With continued reference to FIG. 1, computing device 104 may be configured to generate a classifier using a K-nearest neighbors (KNN) algorithm. A “K-nearest neighbors algorithm” as used in this disclosure, includes a classification method that utilizes feature similarity to analyze how closely out-of-sample-features resemble training data to classify input data to one or more clusters and/or categories of features as represented in training data; this may be performed by representing both training data and input data in vector forms, and using one or more measures of vector similarity to identify classifications within training data, and to determine a classification of input data. K-nearest neighbors' algorithm may include specifying a K-value, or a number directing the classifier to select the k most similar entries training data to a given sample, determining the most common classifier of the entries in the database, and classifying the known sample; this may be performed recursively and/or iteratively to generate a classifier that may be used to classify input data as further samples. For instance, an initial set of samples may be performed to cover an initial heuristic and/or “first guess” at an output and/or relationship, which may be seeded, without limitation, using expert input received according to any process as described herein. As a non-limiting example, an initial heuristic may include a ranking of associations between inputs and elements of training data. Heuristic may include selecting some number of highest-ranking associations and/or training data elements.

With continued reference to FIG. 1, generating k-nearest neighbors' algorithm may generate a first vector output containing a data entry cluster, generating a second vector output containing an input data, and calculate the distance between the first vector output and the second vector output using any suitable norm such as cosine similarity, Euclidean distance measurement, or the like. Each vector output may be represented, without limitation, as an n-tuple of values, where n is at least two values. Each value of n-tuple of values may represent a measurement or other quantitative value associated with a given category of data, or attribute, examples of which are provided in further detail below; a vector may be represented, without limitation, in n-dimensional space using an axis per category of value represented in n-tuple of values, such that a vector has a geometric direction characterizing the relative quantities of attributes in the n-tuple as compared to each other. Two vectors may be considered equivalent where their directions, and/or the relative quantities of values within each vector as compared to each other, are the same; thus, as a non-limiting example, a vector represented as [5, 10, 15] may be treated as equivalent, for purposes of this disclosure, as a vector represented as [1, 2, 3]. Vectors may be more similar where their directions are more similar, and more different where their directions are more divergent; however, vector similarity may alternatively or additionally be determined using averages of similarities between like attributes, or any other measure of similarity suitable for any n-tuple of values, or aggregation of numerical similarity measures for the purposes of loss functions as described in further detail below. Any vectors as described herein may be scaled, such that each vector represents each attribute along an equivalent scale of values. Each vector may be “normalized,” or divided by a “length” attribute, such as a length attribute l as derived using a Pythagorean norm: l=\sqrt{Σᵢ₌₀ⁿ aᵢ²}, where aᵢ is attribute number i of the vector. Scaling and/or normalization may function to make vector comparison independent of absolute quantities of attributes, while preserving any dependency on similarity of attributes; this may, for instance, be advantageous where cases represented in training data are represented by different quantities of samples, which may result in proportionally equivalent vectors with divergent values.

With continued reference to FIG. 1, computing device 104 locates a nutrient 128 intended to address a measurement 124. A “nutrient,” as used in this disclosure, is an eating plan intended to treat, prevent, reverse, and/or cure a geriatric process. A nutrient 128 may map out and identify meals that a user is recommended to consume. A nutrient 128 may identify a list of foods that a user should avoid consuming as they may worsen a user's geriatric process 108. A nutrient 128 may identify a list of foods that a user should consume frequently as they may aid in treating, preventing, and/or reversing a user's geriatric process 108. A nutrient 128 may identify a list of foods that a user may be recommended to consume in moderation. A nutrient 128 may contain information such as a recommended serving size and ingredients that can be included in a particular meal. For instance, and without limitation, a nutrient 128 may recommend a user with a geriatric process 108 such as osteoarthritis to consume a diet full of anti-inflammatory Mediterranean style meals such as a breakfast consisting of full fat Greek yogurt with nuts, fruit, and honey, a lunch consisting of bean stew, and a dinner consisting of an omelet with feta accompanied by a simple salad. In yet another non-limiting example, a nutrient 128 may recommend a user with a geriatric process 108 such as vision loss to consume a breakfast consisting of an egg white scramble, a lunch consisting of sweet potato Mediterranean chili, and a dinner consisting of spaghetti squash noodles loaded with tomato sauce and ground turkey sausage.

With continued reference to FIG. 1, computing device 104 may locate a nutrient 128 by training a machine learning process 132 using a training set 136. A “machine learning process,” as used in this disclosure, is a process that automatically uses training data to generate an algorithm that will be performed by computing device 104 to produce outputs given data provided as inputs; this is in contrast to a non-machine learning software program where the commands to be executed are determined in advance by a user and written in a programming language. A machine learning process 132 may be trained using a training set 136. A “training set,” as used in this disclosure is data containing correlations that a machine learning process may use to model relationships between two or more categories of data elements. For instance, and without limitation, training set 136 may include a plurality of data entries, relating psychiatric markers and nutrient variations to nourishment programs. Training set 136 may be obtained from one or more sources, including for example expert input, public forums, publications, and the like. Information pertaining to training set 136 may be contained within psychiatric database. In an embodiment, machine learning process 132 may utilize a measurement 124 as an input and output a nutrient 128.

With continued reference to FIG. 1, computing device 104 may determine a nutrient interval. A “nutrient interval,” as used in this disclosure, is a recommended optimal time window as to when a meal and/or ingredient contained within a nutrient 128 should be consumed. A time window may identify a particular meal and/or optimal timing of consumption of a meal. For instance, and without limitation, a nutrient interval may specify that a user is recommended to consume a breakfast consisting of steel cut oatmeal with mixed berries between the hours of 7 am-8 am on a Wednesday morning. In yet another non-limiting example, a nutrient interval may specify that a user is recommended to consume a lunch containing 25 grams of carbohydrates and 45 grams of protein at 12:15 pm for optimal digestion. Information pertaining to nutrient interval may be stored and contained within user database 112. Computing device 104 may utilize one or more machine learning processes to calculate a nutrient interval. In an embodiment, machine learning process 132 may output a nutrient interval in conjunction with a nutrient 128. This may be performed utilizing any methodology as described herein.

With continued reference to FIG. 1, computing device 104 may generate a remedy. A “remedy,” as used in this disclosure, is a treatment that aids in treating, preventing, curing, and/or reversing a geriatric process 108. A remedy may include one or more holistic recommendations including but not limited to spiritual treatments, stress reduction techniques, prescription medications, herbal remedies, supplements, naturopathic treatments, over the counter medications, fitness treatments, recommend exercises, physical therapy, occupational therapy, yoga, meditation, tai chi, chi gong, durable medical equipment, and the like. For instance, and without limitation, a remedy may include a recommendation such as a series of logic games for a user suffering from a geriatric process 108 such as mild cognitive dysfunction. In yet another non-limiting example, a remedy may include a recommendation such as a series of stretching exercises to be performed for a user with a geriatric process 108 such as a stiff back. Computing device 104 may generate a remedy using a machine learning process whereby computing device 104 may train a machine learning process to utilize nutrient 128 and measurement 124 as an input and output a remedy. This may be performed utilizing any methodology as described herein. Computing device 104 may locate a plurality of remedies relating to a nutrient 128 and geriatric process 108. Computing device 104 may select a first remedy as a function of a user's effective age 116. For instance, and without limitation, computing device 104 may select a first remedy such as a fitness routine for a user with an effective age of 52, while computing device 104 may select a first remedy such as a supplement for a user with an effective age of 82. Information pertaining to remedies may be stored in user database 112 and/or nutrient database 140.

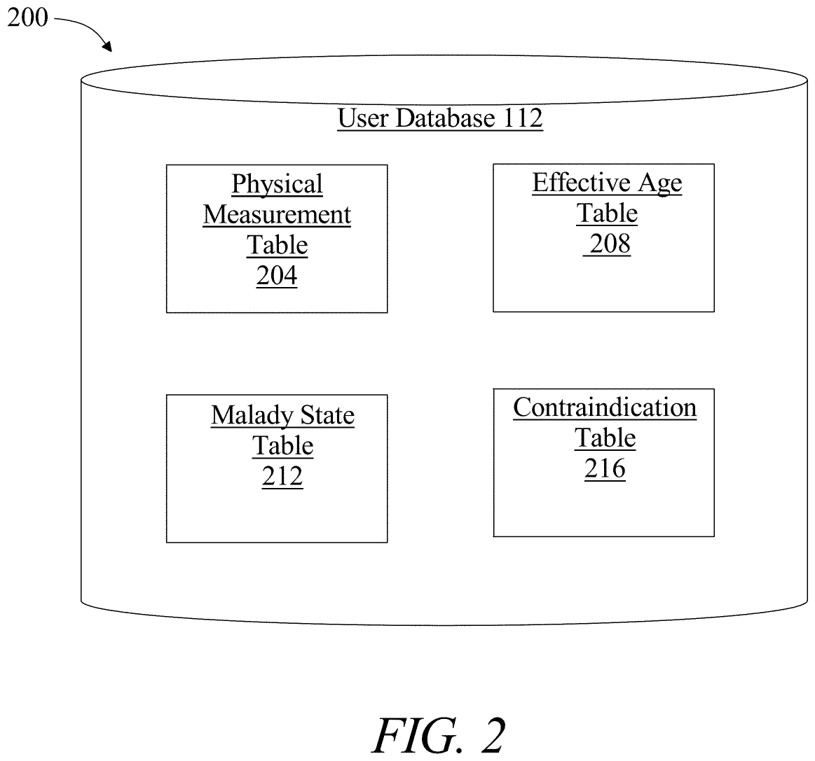

Referring now to FIG. 2, an exemplary embodiment 200 of user database 112 is illustrated. One or more tables contained within user database 112 may include physical measurement table 204; physical measurement table 204 may contain one or more physical measurements pertaining to a user. For instance, and without limitation, physical measurement table 204 may include a questionnaire describing a user's current mental health status. One or more tables contained within user database 112 may include effective age table 208; effective age table 208 may include information describing a user's effective age. For instance, and without limitation, effective age table 208 may indicate that a user has a chronological age of 88 and an effective age of 92. One or more tables contained within user database 112 may include malady state table 212; malady state table 212 may include one or more maladies that a user currently suffers from. For instance, and without limitation, malady state table 212 may specify that a user suffers from a diagnosed medical condition such as hypertension. One or more tables contained within user database 112 may include contraindication table 216; contraindication table 216 may include one or more ingredients and/or remedies that may be contraindicated for a user. For instance, and without limitation, contraindication table 216 may specify that a user is allergic to an ingredient such as tree nuts.

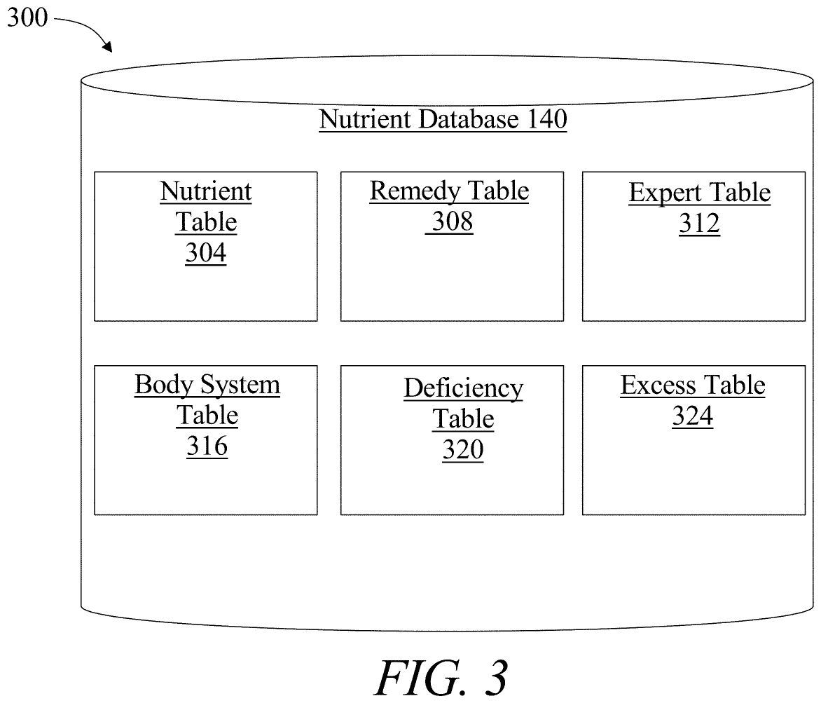

Referring now to FIG. 3, an exemplary embodiment 300 of nutrient database 140 is illustrated. One or more tables contained within nutrient database 140 may include nutrient table 304; nutrient table 304 may include information relating to one or more nutrients. For instance, and without limitation, nutrient table 304 may describe one or more ingredients contained within a meal consisting of chicken fajitas. One or more tables contained within nutrient database 140 may include remedy table 308; remedy table 308 may include information relating to one or more remedies. For instance, and without limitation, remedy table 308 may describe remedies available for a geriatric process such as migraines. One or more tables contained within nutrient database 140 may include expert table 312; expert table 312 may include one or more expert inputs. For instance, and without limitation, expert table 312 may include a journal article describing measurements associated with a geriatric process such as hearing loss. One or more tables contained within nutrient database 140 may include body system table 316; body system table 316 may include body systems associated with various geriatric processes. For instance, and without limitation, body system table 316 may specify that a geriatric process such as nail fungus may be associated with a body system that includes the immune system. One or more tables contained within nutrient database 140 may include deficiency table 320; deficiency table 320 may include one or more entries relating to a deficiency. For instance, and without limitation, deficiency table 320 may indicate that an iron level below 10 g/dL for a female may indicate a deficiency. One or more tables contained within nutrient database 140 may include excess table 324; excess table 324 may include one or more entries relating to an excess. For instance, and without limitation, excess table 324 may indicate that a morning cortisol level above 25 mcg/dL may indicate an excess.

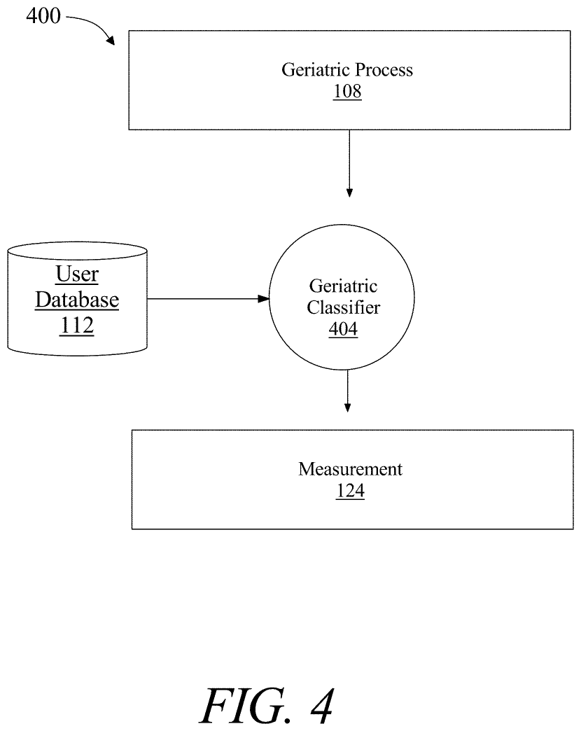

Referring now to FIG. 4, an exemplary embodiment 400 of geriatric classifier 404 is illustrated. Geriatric classifier 404 may be implemented using any methodology as described above in more detail in reference to FIG. 1. Geriatric classifier 404 may be trained using a training set that relates a plurality of geriatric processes to a plurality of measurements. Training set may include any training set and/or training data as described herein. Geriatric classifier 404 may utilize a geriatric process 108 relating to a user as an input and output a measurement 124 using a classification process. Classification process may include one or more classification algorithms as described above in more detail in reference to FIG. 1. In an embodiment, information pertaining to a geriatric process 108 may be stored within user database 112.

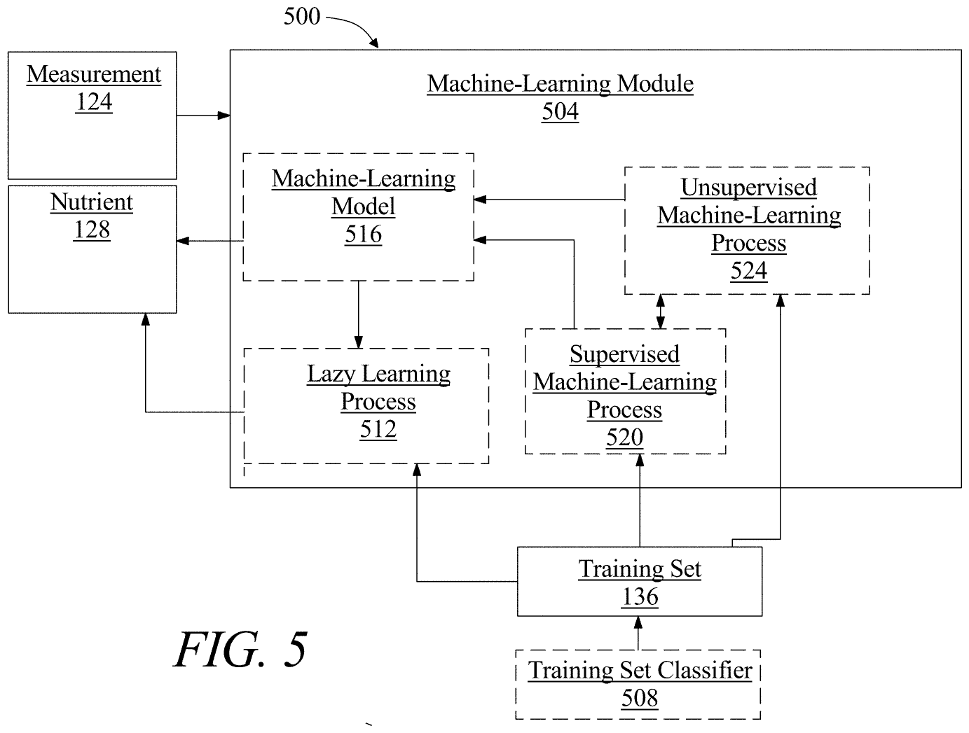

Referring now to FIG. 5, an exemplary embodiment 500 of a machine learning module 504 that may perform one or more machine learning processes as described in this disclosure is illustrated. Machine learning module may perform determinations, classification, and/or analysis steps, methods, processes, or the like as described in this disclosure using machine learning processes. Machine learning module may produce outputs such as nutrients 128 given data provided as inputs such as measurement 124; this is in contrast to a non-machine learning software program where the commands to be executed are determined in advance by a user and written in a programming language.

Further referring to FIG. 5, training set 136 may be filtered, sorted, and/or selected using one or more supervised and/or unsupervised machine learning processes and/or models as described in further detail below; such models may include without limitation a training data classifier 508. Training data classifier 508 may include a classifier, which is a machine learning model as defined above, such as a mathematical model, neural net, or program generated by a machine learning algorithm known as a “classification algorithm,” as described in further detail below, that sorts inputs into categories or bins of data, outputting the categories or bins of data and/or labels associated therewith. A classifier may be configured to output at least a datum that labels or otherwise identifies a set of data that are clustered together, found to be close under a distance metric as described below, or the like. Machine learning module 504 may generate a classifier using a classification algorithm, defined as a process whereby a computing device and/or any module and/or component operating thereon derives a classifier from training data such as training set 136. Classification may be performed using, without limitation, linear classifiers such as without limitation logistic regression and/or naive Bayes classifiers, nearest neighbor classifiers such as k-nearest neighbors' classifiers, support vector machines, least squares support vector machines, fisher's linear discriminant, quadratic classifiers, decision trees, boosted trees, random forest classifiers, learning vector quantization, and/or neural network-based classifiers. As a non-limiting example, training data classifier 508 may classify elements of training data to a cohort of persons having a particular psychiatric disease and/or psychiatric condition such as bipolar disorder or schizophrenia.

Still referring to FIG. 5, machine learning module 504 may be configured to perform a lazy-learning process 512 and/or protocol, which may alternatively be referred to as a “lazy loading” or “call-when-needed” process and/or protocol, may be a process whereby machine learning is conducted upon receipt of an input to be converted to an output, by combining the input and training set to derive the algorithm to be used to produce the output on demand. For instance, an initial set of simulations may be performed to cover an initial heuristic and/or “first guess” at an output and/or relationship. As a non-limiting example, an initial heuristic may include a ranking of associations between inputs and elements of training set 136. Heuristic may include selecting some number of highest-ranking associations and/or training set elements. Lazy learning may implement any suitable lazy learning algorithm, including without limitation a K-nearest neighbors' algorithm, a lazy naïve Bayes algorithm, or the like; persons skilled in the art, upon reviewing the entirety of this disclosure, will be aware of various lazy-learning algorithms that may be applied to generate outputs as described in this disclosure, including without limitation lazy learning applications of machine learning algorithms as described in further detail below.

Alternatively, or additionally, and with continued reference to FIG. 5, machine learning processes as described in this disclosure may be used to generate machine learning models 516. A “machine learning model,” as used in this disclosure, is a mathematical and/or algorithmic representation of a relationship between inputs and outputs, as generated using any machine learning process including without limitation any process as described above and stored in memory; an input is submitted to a machine learning model 516 once created, which generates an output based on the relationship that was derived. For instance, and without limitation, a linear regression model, generated using a linear regression algorithm, may compute a linear combination of input data using coefficients derived during machine learning processes to calculate an output datum. As a further non-limiting example, a machine learning model 516 may be generated by creating an artificial neural network, such as a convolutional neural network comprising an input layer of nodes, one or more intermediate layers, and an output layer of nodes. Connections between nodes may be created via the process of “training” the network, in which elements from a training set 136 set are applied to the input nodes, a suitable training algorithm (such as Levenberg-Marquardt, conjugate gradient, simulated annealing, or other algorithms) is then used to adjust the connections and weights between nodes in adjacent layers of the neural network to produce the desired values at the output nodes. This process is sometimes referred to as deep learning.

Still referring to FIG. 5, machine learning algorithms may include at least a supervised machine learning process 520. At least a supervised machine learning process 520, as defined herein, include algorithms that receive a training set relating a number of inputs to a number of outputs, and seek to find one or more mathematical relations relating inputs to outputs, where each of the one or more mathematical relations is optimal according to some criterion specified to the algorithm using some scoring function. For instance, a supervised learning algorithm may include measurement 124 as described above as inputs, nutrient 128 as outputs, and a scoring function representing a desired form of relationship to be detected between inputs and outputs; scoring function may, for instance, seek to maximize the probability that a given input and/or combination of elements inputs is associated with a given output to minimize the probability that a given input is not associated with a given output. Scoring function may be expressed as a risk function representing an “expected loss” of an algorithm relating inputs to outputs, where loss is computed as an error function representing a degree to which a prediction generated by the relation is incorrect when compared to a given input-output pair provided in training set 136. Persons skilled in the art, upon reviewing the entirety of this disclosure, will be aware of various possible variations of at least a supervised machine learning process 520 that may be used to determine relation between inputs and outputs. Supervised machine learning processes may include classification algorithms as defined above.

Further referring to FIG. 5, machine learning processes may include at least an unsupervised machine learning process 524. An unsupervised machine learning process, as used herein, is a process that derives inferences in datasets without regard to labels; as a result, an unsupervised machine learning process may be free to discover any structure, relationship, and/or correlation provided in the data. Unsupervised processes may not require a response variable; unsupervised processes may be used to find interesting patterns and/or inferences between variables, to determine a degree of correlation between two or more variables, or the like.

Still referring to FIG. 5, machine learning module 504 may be designed and configured to create a machine learning model 516 using techniques for development of linear regression models. Linear regression models may include ordinary least squares regression, which aims to minimize the square of the difference between predicted outcomes and actual outcomes according to an appropriate norm for measuring such a difference (e.g., a vector-space distance norm); coefficients of the resulting linear equation may be modified to improve minimization. Linear regression models may include ridge regression methods, where the function to be minimized includes the least-squares function plus term multiplying the square of each coefficient by a scalar amount to penalize large coefficients. Linear regression models may include least absolute shrinkage and selection operator (LASSO) models, in which ridge regression is combined with multiplying the least-squares term by a factor of 1 divided by double the number of samples. Linear regression models may include a multi-task lasso model wherein the norm applied in the least-squares term of the lasso model is the Frobenius norm amounting to the square root of the sum of squares of all terms. Linear regression models may include the elastic net model, a multi-task elastic net model, a least angle regression model, a LARS lasso model, an orthogonal matching pursuit model, a Bayesian regression model, a logistic regression model, a stochastic gradient descent model, a perceptron model, a passive aggressive algorithm, a robustness regression model, a Huber regression model, or any other suitable model that may occur to persons skilled in the art upon reviewing the entirety of this disclosure. Linear regression models may be generalized in an embodiment to polynomial regression models, whereby a polynomial equation (e.g., a quadratic, cubic or higher-order equation) providing a best predicted output/actual output fit is sought; similar methods to those described above may be applied to minimize error functions, as will be apparent to persons skilled in the art upon reviewing the entirety of this disclosure.

Continuing to refer to FIG. 5, machine learning algorithms may include, without limitation, linear discriminant analysis. Machine learning algorithm may include quadratic discriminate analysis. Machine learning algorithms may include kernel ridge regression. Machine learning algorithms may include support vector machines, including without limitation support vector classification-based regression processes. Machine learning algorithms may include stochastic gradient descent algorithms, including classification and regression algorithms based on stochastic gradient descent. Machine learning algorithms may include nearest neighbors' algorithms. Machine learning algorithms may include Gaussian processes such as Gaussian Process Regression. Machine learning algorithms may include cross-decomposition algorithms, including partial least squares and/or canonical correlation analysis. Machine learning algorithms may include naïve Bayes methods. Machine learning algorithms may include algorithms based on decision trees, such as decision tree classification or regression algorithms. Machine learning algorithms may include ensemble methods such as bagging meta-estimator, forest of randomized tress, AdaBoost, gradient tree boosting, and/or voting classifier methods. Machine learning algorithms may include neural net algorithms, including convolutional neural net processes.

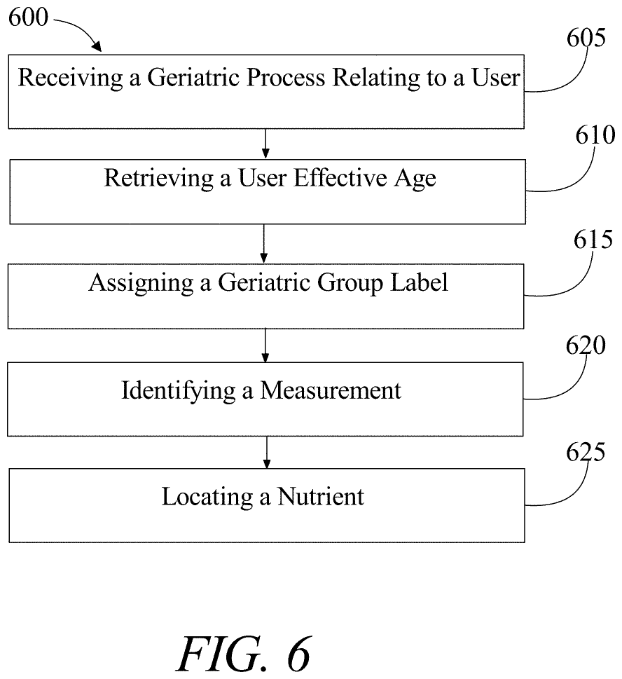

Referring now to FIG. 6, an exemplary embodiment 600 of a method of reversing a geriatric process is illustrated. At step 605, computing device 104 receives a geriatric process 108 relating to a user. A geriatric process 108 may include any of the geriatric processes 108 as described above in more detail in reference to FIG. 1. For example, a geriatric process 108 may include a description of an episode of shortness of breath and fatigue that a user may experience when the user exerts himself. In yet another non-limiting example, a geriatric process 108 may include a description of dizziness and a feeling of imbalance upon standing. Information pertaining to a geriatric process 108 may be stored within user database 112. Computing device 104 may receive a geriatric process 108 using any network methodology as described herein.

With continued reference to FIG. 6, at step 610, computing device 104 retrieves a user effective age 116. An effective age 116 includes any effective age 116 as described above in more detail in reference to FIG. 1.

With continued reference to FIG. 6, at step 615, computing device 104 assigns a geriatric process 108 a geriatric group label 120 as a function of a user effective age 116. A geriatric group label 120, includes any of the geriatric group labels 120 as described above in more detail in reference to FIG. 1. A geriatric group label 120 may identify a root cause of a geriatric process 108. For instance, and without limitation, a geriatric group label 120 may identify a geriatric process 108 such as Type 2 Diabetes Mellitus as having a root cause such as living a sedentary lifestyle. Information pertaining to a geriatric group label 120 may be stored and contained within user database 112 and/or nutrient database 140. In an embodiment, a geriatric group label 120 may identify a body system as described above in more detail in reference to FIG. 1. For instance, and without limitation, a geriatric group label 120 may identify that a geriatric process 108 such as recurrent yeast infections may be related to the gastrointestinal body system, due to a disbalance of beneficial bacteria. Computing device 104 may assign a geriatric group label 120 utilizing effective age 116. In an embodiment, computing device 104 may subtract a user effective age 116 from a user chronological age and output a deficit. Deficit may include any deficit as described above in more detail in reference to FIG. 1. Computing device 104 may assign a geriatric group label 120 as a function of a deficit. For instance, and without limitation, a user with a chronological age of 75 and an effective age of 52 may produce a deficit of 23. In yet another non-limiting example, a user with a chronological age of 82 and an effective age of 102 may produce a deficit of negative 20. Computing device 104 may utilize deficits to assign a geriatric group label 120. This may be performed utilizing any methodology as described above in more detail in reference to FIG. 1. In an embodiment, computing device 104 may locate a malady state associated with a user. A malady state includes any malady state as described above in more detail in reference to FIG. 1. For example, a malady state may indicate that a user was previously diagnosed with a medical condition such as Chron's disease. Computing device 104 may compare a malady state to a geriatric process 108 and assign a geriatric group label 120 as a function of the comparison. For instance, and without limitation, computing device 104 may compare a malady state such as rheumatoid arthritis to a geriatric process 108 such as lupus and assign a geriatric group label 120 that indicates autoimmune dysfunction.

With continued reference to FIG. 6, at step 620, computing device 104 identifies a measurement 124 relating to a geriatric group label 120. A measurement 124 includes any of the measurements as described above in more detail in reference to FIG. 1. For instance, and without limitation, a measurement 124 may include a blood sample analyzed for levels of nutrients such as calcium and magnesium. In an embodiment, a measurement 124 may indicate a deficiency and/or an excess, as described above in more detail in reference to FIG. 1. In an embodiment, computing device 104 may identify a measurement 124 utilizing a geriatric classifier. Geriatric classifier may include any classifier as described above in more detail in reference to FIGS. 1-5. Geriatric classifier may be trained using a training set that relates a plurality of geriatric processes to a plurality of measurements. Geriatric classifier may input a geriatric process 108 relating to a user and output a measurement 124 as a function of a classification process.

With continued reference to FIG. 6, at step 625, computing device 104 locates a nutrient 128 intended to address a measurement 124. Locating a nutrient 128 may include training a machine learning process 132 as a function of a training set 136, wherein the training set 136 may relate a plurality of measurements to a plurality of nutrients. Computing device 104 may locate a nutrient 128 as a function of a measurement 124 and a machine learning process 132. This may be performed utilizing any methodology as described above in more detail in reference to FIGS. 1-5. Nutrient 128 includes any nutrient as described above in more detail in reference to FIG. 1. In an embodiment, nutrient 128 may include a nutrition plan intended to treat, prevent, reverse, and/or cure a geriatric process 108. Computing device 104 may determine a nutrient interval, such as a recommended day and/or time when a particular ingredient and/or meal is best recommended to be consumed. In an embodiment, computing device 104 may generate a remedy in addition to a nutrient 128. A remedy includes any remedy as described above in more detail in reference to FIGS. 1-5. In an embodiment, a remedy may include a recommended exercise for a user to perform. In an embodiment, a remedy may include a recommended stress reduction technique such as a meditation sequence to be performed nightly for twenty minutes. Computing device 104 may select a first remedy as a function of a user effective age 116 as described above in more detail in reference to FIG. 1.

It is to be noted that any one or more of the aspects and embodiments described herein may be conveniently implemented using one or more machines (e.g., one or more computing devices that are utilized as a user computing device for an electronic document, one or more server devices, such as a document server, etc.) programmed according to the teachings of the present specification, as will be apparent to those of ordinary skill in the computer art. Appropriate software coding can readily be prepared by skilled programmers based on the teachings of the present disclosure, as will be apparent to those of ordinary skill in the software art. Aspects and implementations discussed above employing software and/or software modules may also include appropriate hardware for assisting in the implementation of the machine executable instructions of the software and/or software module.

Such software may be a computer program product that employs a machine-readable storage medium. A machine-readable storage medium may be any medium that is capable of storing and/or encoding a sequence of instructions for execution by a machine (e.g., a computing device) and that causes the machine to perform any one of the methodologies and/or embodiments described herein. Examples of a machine-readable storage medium include, but are not limited to, a magnetic disk, an optical disc (e.g., CD, CD-R, DVD, DVD-R, etc.), a magneto-optical disk, a read-only memory “ROM” device, a random-access memory “RAM” device, a magnetic card, an optical card, a solid-state memory device, an EPROM, an EEPROM, and any combinations thereof. A machine-readable medium, as used herein, is intended to include a single medium as well as a collection of physically separate media, such as, for example, a collection of compact discs or one or more hard disk drives in combination with a computer memory. As used herein, a machine-readable storage medium does not include transitory forms of signal transmission.

Such software may also include information (e.g., data) carried as a data signal on a data carrier, such as a carrier wave. For example, machine-executable information may be included as a data-carrying signal embodied in a data carrier in which the signal encodes a sequence of instruction, or portion thereof, for execution by a machine (e.g., a computing device) and any related information (e.g., data structures and data) that causes the machine to perform any one of the methodologies and/or embodiments described herein.

Examples of a computing device include, but are not limited to, an electronic book reading device, a computer workstation, a terminal computer, a server computer, a handheld device (e.g., a tablet computer, a smartphone, etc.), a web appliance, a network router, a network switch, a network bridge, any machine capable of executing a sequence of instructions that specify an action to be taken by that machine, and any combinations thereof. In one example, a computing device may include and/or be included in a kiosk.

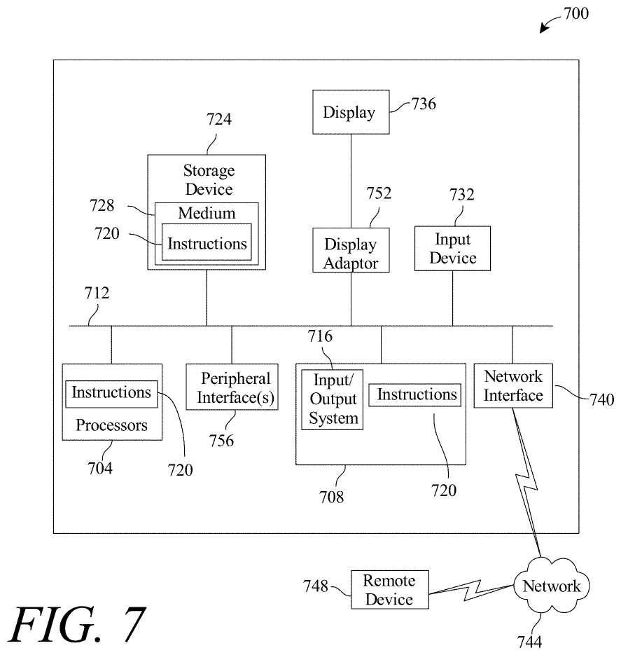

FIG. 7 shows a diagrammatic representation of one embodiment of a computing device in the exemplary form of a computer system 700 within which a set of instructions for causing a control system to perform any one or more of the aspects and/or methodologies of the present disclosure may be executed. It is also contemplated that multiple computing devices may be utilized to implement a specially configured set of instructions for causing one or more of the devices to perform any one or more of the aspects and/or methodologies of the present disclosure. Computer system 700 includes a processor 704 and a memory 708 that communicate with each other, and with other components, via a bus 712. Bus 712 may include any of several types of bus structures including, but not limited to, a memory bus, a memory controller, a peripheral bus, a local bus, and any combinations thereof, using any of a variety of bus architectures.

Memory 708 may include various components (e.g., machine-readable media) including, but not limited to, a random-access memory component, a read only component, and any combinations thereof. In one example, a basic input/output system 716 (BIOS), including basic routines that help to transfer information between elements within computer system 700, such as during start-up, may be stored in memory 708. Memory 708 may also include (e.g., stored on one or more machine-readable media) instructions (e.g., software) 720 embodying any one or more of the aspects and/or methodologies of the present disclosure. In another example, memory 708 may further include any number of program modules including, but not limited to, an operating system, one or more application programs, other program modules, program data, and any combinations thereof.

Computer system 700 may also include a storage device 724. Examples of a storage device (e.g., storage device 724) include, but are not limited to, a hard disk drive, a magnetic disk drive, an optical disc drive in combination with an optical medium, a solid-state memory device, and any combinations thereof. Storage device 724 may be connected to bus 712 by an appropriate interface (not shown). Example interfaces include, but are not limited to, SCSI, advanced technology attachment (ATA), serial ATA, universal serial bus (USB), IEEE 1394 (FIREWIRE), and any combinations thereof. In one example, storage device 724 (or one or more components thereof) may be removably interfaced with computer system 700 (e.g., via an external port connector (not shown)). Particularly, storage device 724 and an associated machine-readable medium 728 may provide nonvolatile and/or volatile storage of machine-readable instructions, data structures, program modules, and/or other data for computer system 700. In one example, software 720 may reside, completely or partially, within machine-readable medium 728. In another example, software 720 may reside, completely or partially, within processor 704.

Computer system 700 may also include an input device 732. In one example, a user of computer system 700 may enter commands and/or other information into computer system 700 via input device 732. Examples of an input device 732 include, but are not limited to, an alpha-numeric input device (e.g., a keyboard), a pointing device, a joystick, a gamepad, an audio input device (e.g., a microphone, a voice response system, etc.), a cursor control device (e.g., a mouse), a touchpad, an optical scanner, a video capture device (e.g., a still camera, a video camera), a touchscreen, and any combinations thereof. Input device 732 may be interfaced to bus 712 via any of a variety of interfaces (not shown) including, but not limited to, a serial interface, a parallel interface, a game port, a USB interface, a FIREWIRE interface, a direct interface to bus 712, and any combinations thereof. Input device 732 may include a touch screen interface that may be a part of or separate from display 736, discussed further below. Input device 732 may be utilized as a user selection device for selecting one or more graphical representations in a graphical interface as described above.

A user may also input commands and/or other information to computer system 700 via storage device 724 (e.g., a removable disk drive, a flash drive, etc.) and/or network interface device 740. A network interface device, such as network interface device 740, may be utilized for connecting computer system 700 to one or more of a variety of networks, such as network 744, and one or more remote devices 748 connected thereto. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network, such as network 744, may employ a wired and/or a wireless mode of communication. In general, any network topology may be used. Information (e.g., data, software 720, etc.) may be communicated to and/or from computer system 700 via network interface device 740.

Computer system 700 may further include a video display adapter 752 for communicating a displayable image to a display device, such as display device 736. Examples of a display device include, but are not limited to, a liquid crystal display (LCD), a cathode ray tube (CRT), a plasma display, a light emitting diode (LED) display, and any combinations thereof. Display adapter 752 and display device 736 may be utilized in combination with processor 704 to provide graphical representations of aspects of the present disclosure. In addition to a display device, computer system 700 may include one or more other peripheral output devices including, but not limited to, an audio speaker, a printer, and any combinations thereof. Such peripheral output devices may be connected to bus 712 via a peripheral interface 756. Examples of a peripheral interface include, but are not limited to, a serial port, a USB connection, a FIREWIRE connection, a parallel connection, and any combinations thereof.

The foregoing has been a detailed description of illustrative embodiments of the invention. Various modifications and additions can be made without departing from the spirit and scope of this invention. Features of each of the various embodiments described above may be combined with features of other described embodiments as appropriate in order to provide a multiplicity of feature combinations in associated new embodiments. Furthermore, while the foregoing describes a number of separate embodiments, what has been described herein is merely illustrative of the application of the principles of the present invention. Additionally, although particular methods herein may be illustrated and/or described as being performed in a specific order, the ordering is highly variable within ordinary skill to achieve methods, systems, and software according to the present disclosure. Accordingly, this description is meant to be taken only by way of example, and not to otherwise limit the scope of this invention.

Exemplary embodiments have been disclosed above and illustrated in the accompanying drawings. It will be understood by those skilled in the art that various changes, omissions and additions may be made to that which is specifically disclosed herein without departing from the spirit and scope of the present invention.

What is claimed is:

1. A system for reversing a geriatric process, the system comprising:
a computing device the computing device designed and configured to:
receive, a geriatric process relating to a user;
retrieve a user effective age;
assign the geriatric process a geriatric group label as a function of the user effective age;
identify a measurement relating to the geriatric group label; and
locate a nutrient intended to address the measurement, wherein locating the nutrient further comprises:
training a machine learning process as a function of a training set, wherein the training set relates a plurality of measurements to a plurality of nutrients; and
locating the nutrient as a function of the measurement and the machine learning process.

2. The system of claim 1, wherein the computing device is further configured to:
subtract the user effective age from a user chronological age and output a deficit; and
assign the geriatric group label as a function of the deficit.

3. The system of claim 1, wherein the geriatric group label identifies a body system.

4. The system of claim 1, wherein assigning the geriatric group label further comprises:
locating a malady state associated with the user;
comparing the malady state to the geriatric process; and
assigning the geriatric group label as a function of the comparison.

5. The system of claim 1, wherein identifying the measurement further comprises:
generating a geriatric classifier wherein the geriatric classifier is trained using a training set that relates a plurality of geriatric processes to a plurality of measurements and the geriatric classifier is configured to input the geriatric process relating to the user and output the measurement as a function of a classification process.

6. The system of claim 1, wherein the measurement identifies a deficiency.

7. The system of claim 1, wherein the measurement identifies an excess.

8. The system of claim 1 further comprising:
generate a remedy wherein generating the remedy further comprises:
training a machine learning process wherein the machine learning process utilizes the nutrient and the measurement as an input, and outputs the remedy.

9. The system of claim 9, wherein generating the remedy further comprises:
locating a plurality of remedies relating to the nutrient and the geriatric process; and
selecting a first remedy as a function of the user effective age.

10. The system of claim 1 further comprising determining a nutrient interval.

11. A method of reversing a geriatric process, the method comprising:
receiving by a computing device, a geriatric process relating to a user;
retrieving by the computing device, a user effective age;
assigning the geriatric process, by the computing device, a geriatric group label as a function of the user effective age;
identifying by the computing device, a measurement relating to the geriatric group label; and
locating by the computing device, a nutrient intended to address the measurement, wherein
locating the nutrient further comprises:
training a machine learning process as a function of a training set, wherein the training set relates a plurality of measurements to a plurality of nutrients; and
locating the nutrient as a function of the measurement and the machine learning process.

12. The method of claim 11, wherein assigning the geriatric group label further comprises:
subtracting the user effective age from a user chronological age and output a deficit; and
assigning the geriatric group label as a function of the deficit.

13. The method of claim 11, wherein the geriatric group label identifies a body system.

14. The method of claim 11, wherein assigning the geriatric group label further comprises:
locating a malady state associated with the user;
comparing the malady state to the geriatric process; and
assigning the geriatric group label as a function of the comparison.

15. The method of claim 11, wherein identifying the measurement further comprises:
generating a geriatric classifier wherein the geriatric classifier is trained using a training set that relates a plurality of geriatric processes to a plurality of measurements and the geriatric classifier is configured to input the geriatric process relating to the user and output the measurement as a function of a classification process.

16. The method of claim 11, wherein the measurement identifies a deficiency.

17. The method of claim 11, wherein the measurement identifies an excess.

18. The method of claim 11 further comprising:
generating a remedy wherein generating the remedy further comprises:
training a machine learning process wherein the machine learning process utilizes the nutrient and the measurement as an input, and outputs the remedy.

19. The method of claim 19, wherein generating the remedy further comprises:
locating a plurality of remedies relating to the nutrient and the geriatric process; and
selecting a first remedy as a function of the user effective age.

20. The method of claim 11 further comprising determining a nutrient interval.
